(19) United States
(12) Patent Application Publication
(10) Pub. No.: US 2023/0089501 A1
(43) Pub. Date: Mar. 23, 2023

(54) SANITIZING DEVICE WITH PATHOGEN DETECTION, SANITIZING SYSTEM WITH PATHOGEN DETECTION, AND METHODS FOR USE THEREOF

(71) Applicant: Population Lights, Inc., San Francisco, CA (US)

(72) Inventor: Megan Groves, San Francisco, CA (US)

(21) Appl. No.: 18/070,142

(22) Filed: Nov. 28, 2022

Related U.S. Application Data
(63) Continuation of application No. 17/548,477, filed on Dec. 10, 2021, now abandoned.
Application No. 17/548,477 is a continuation of application No. 17/332,947, filed on May 27, 2021, now abandoned.
(60) Provisional application No. 63/075,197, filed on Sep. 6, 2020.

(51) Int. Cl. A61L 9/18; A61L 2/08; A61L 2/24

(52) U.S. Cl. CPC A61L 9/18; A61L 2/08; A61L 2/24; A61L 2209/111; A61L 2202/25

(57) ABSTRACT

Sanitizing devices may be configured to emit sanitizing light into an environment in order to sanitize air, objects, and/or surfaces in the environment. The sanitizing light may be of a wavelength, or set of wavelengths, that deactivates, kills, or neutralizes, pathogens such as bacteria and viruses but, is not harmful to human or animal tissue. The sanitizing devices may be configured to be able to communicate with each other and/or a user over, for example, the Internet, a wireless communication network, a mesh network, and/or a near-field communication protocol in order to, for example, provide operation information and/or receive operation instructions.

### RELATED APPLICATION

This patent application is Continuation of U.S. Non-Provisional patent application Ser. No. 17/548,477, filed on 10 Dec. 2021, which is a Continuation of U.S. Non-Provisional patent application Ser. No. 17/332,947, filed on 27 May 2021, which is a Non-Provisional Patent application claiming priority to U.S. Provisional Patent Application No. 63/075,197, filed on 6 Sep. 2020 and entitled “SANITIZING DEVICE, SANITIZING SYSTEM, AND METHODS FOR USE THEREOF,” both of which are incorporated in their entirety herein.

### BACKGROUND

Ultraviolet light has been used in certain industrial and medical settings to destroy pathogens (e.g., bacteria and viruses) and sanitize surfaces and objects. However, a drawback with these technologies is that the wavelengths of most ultraviolet light used cause harm to humans and animals, particularly to their eyes and skin. This safety concern makes wide-scale adoption of this sanitization method difficult.

In addition traditional methods of sanitizing air, objects, and/or surfaces are typically operated as so called “dumb devices” in that they are not Internet enabled and may not be controlled remotely or via a computer software application running on, for example, a server or mobile software application. do not offer sophisticated operation

### SUMMARY

Sanitizing devices disclosed herein may include a power source, a sanitizing light source, a control system, and a housing. The power source may be electrically coupled to the to the sanitizing light source, and the control system and configured to provide electrical power thereto via, for example, a battery, which may be rechargeable or otherwise. Additionally, or alternatively, power source may be coupled to a power cord configured to couple to an electrical main. On some occasions, power source may couple to an electrical main via a port configured for acceptance of a power cord therein.

The sanitizing light source may be configured to emit sanitizing light into an environment through an aperture in a housing that houses the sanitizing light source, the control system, and the power source responsively to an instruction from the control system. The sanitizing light may have a wavelength within the range of, for example, 200-240 nm, 210-280 nm and/or 390-420 nm.

In some embodiments, the sanitizing light source may be configured to emit the sanitizing light toward one or more reflectors or sensors arranged within the environment so that, for example, operation of the sanitizing device may be optimized and/or light emitted by sanitizing light source may be directed to one or more desired locations within the environment.

The control system may be communicatively coupled to the sanitizing light source and configured to control an intensity of the sanitizing light emitted by the sanitizing light source and/or trigger an operation of the sanitizing light source.

The housing may be configured to house the power source, the sanitizing light source, and the control system and may include at least one aperture through which sanitizing light emitted by the sanitizing light source may exit the housing into the environment. In some embodiments, the housing may have a truncated-cube-like shape. Additionally, or alternatively, the sanitizing device and/or housing may be configured to be, for example, hung from a ceiling, placed on a surface, and/or mounted on a wall.

In some embodiments, the sanitizing device may include a filter configured to filter sanitizing light emitted by the sanitizing light prior to the sanitizing light exiting via the aperture so that the sanitizing light exiting the housing has a wavelength in the range of 200-240 nm, 210-280 nm and/or 390-420 nm.

On some occasions, the sanitizing device may include a sensor that may be configured to monitor a feature of the environment and communicate a result of the monitoring to, for example, the control system and/or an external device such as a server or computer. In some instances, an intensity of the sanitizing light emitted by the sanitizing light source is responsive to receipt of a result of the monitoring performed by the sensor.

In some embodiments, the sanitizing device may further comprise a transceiver configured to communicate information from the control system regarding an operation of the sanitizing device to an external computing device. The transceiver may also be configured to receive instructions regarding its operation via the transceiver.

In some embodiments, the control system and/or a transceiver within and/or in communication with the sanitizing may be configured to communicate with another sanitizing device, control system, and/or transceiver resident within a sanitizing device via a mesh network.

Additionally, or alternatively, the sanitizing device may include a memory communicatively coupled to the control system. The memory may be configured to store, for example, a schedule for an operation of the sanitizing device, information regarding operations of the sanitizing device, information collected by one or more sensors resident in and/or in communication with the sanitizing device, and/or instructions regarding an operation of the sanitizing device.

Exemplary systems disclosed herein may include a plurality of communicatively coupled sanitizing devices that include a power source, a sanitizing light source, a control system, a transceiver configured to communicate with a transceiver resident in another of the sanitizing devices included in the plurality of sanitizing devices and a housing. The system may also include a server communicatively coupled to each of the sanitizing devices included in the plurality of sanitizing devices. The server may be configured to communicate instructions regarding an operation of one or more of the sanitizing devices included in the plurality of sanitizing devices to the respective one or more of the sanitizing devices included in the plurality of sanitizing devices. The instructions communicated to the one or more of the sanitizing devices may include a schedule of operation for each sanitizing device included in the plurality of sanitizing devices.

Communication between components of the system (e.g., between sanitizing devices and/or between a sanitizing device and the server) may be via Internet communications, a mesh network, a near-field communication (NFC) protocol and/or a wireless communication network such as Wi-Fi or BLUETOOTH™.

In some embodiments, the server may be configured to coordinate an operation of one or more of the sanitizing devices included in the plurality of sanitizing devices to collaboratively sanitize one or more objects present in the environment. At times, the coordination may be performed using reflectors positioned within the environment.

In some embodiments, the system may further include a sensor or monitor that is resident within the sanitizing device and/or external to the sanitizing device. The sensor may be configured to monitor a feature and/or aspect of the environment. On some occasions, a result of the monitoring may be communicated to the control system and, in some instances, coordination between two or more components of the system and/or sanitizing devices included within the system may be responsive to the monitoring performed by the sensor.

### BRIEF DESCRIPTION OF THE DRAWINGS

The present invention is illustrated by way of example, and not limitation, in the figures of the accompanying drawings in which:

FIG. 1A is a block diagram of a first exemplary sanitizing device according to the present invention, according to embodiments of the present invention;

FIG. 1B is a block diagram of a second exemplary sanitizing device according to the present invention, according to embodiments of the present invention;

FIGS. 2A-2H provide different views of an exemplary sanitizing devices, according to embodiments of the present invention;

FIGS. 2I-2L provide side plan views of the exemplary sanitizing devices of FIGS. 2A-2H arranged in different orientations in an environment and/or on a surface of an object, according to embodiments of the present invention;

FIG. 3 is a diagram of an exemplary environment in which sanitizing device and/or a sanitizing system may operate to sanitize, or disinfect surfaces, air, and/or objects included therein, according to embodiments of the present invention;

FIG. 4A is a diagram of a first exemplary system deploying a plurality of communicatively connected sanitizing devices, according to embodiments of the present invention;

FIG. 4B is a diagram of a second exemplary system deploying a plurality of communicatively connected sanitizing devices, according to embodiments of the present invention;

FIG. 5 is a flowchart illustrating a process for operating a sanitizing device, according to embodiments of the present invention;

FIG. 6 is a flowchart illustrating a process for centrally operating a plurality of sanitizing devices, according to embodiments of the present invention; and

FIG. 7 is a block diagram of an exemplary processor-based system that may store data and/or execute instructions for the processes disclosed herein, in accordance with some embodiments of the present invention.

Throughout the drawings, the same reference numerals and characters, unless otherwise stated, are used to denote like features, elements, components, or portions of the illustrated embodiments. Moreover, while the subject invention will now be described in detail with reference to the drawings, the description is done in connection with the illustrative embodiments. It is intended that changes and modifications can be made to the described embodiments without departing from the true scope and spirit of the subject invention as defined by the appended claims.

### WRITTEN DESCRIPTION

Disclosed herein are sanitizing devices that emit, or project, light to disinfect, sanitize, clean, or otherwise neutralize pathogens (coronavirus, influenza, E. bola, E. Coli etc.) in the air within and/or on surfaces and objects that may be resident within an environment or enclosed space. The wavelengths of light emitted by the sanitizing devices may be selected so that they are effective at sanitization but do not harm, or adversely impact, human or animal tissue (e.g., eyes or skin). In some embodiments, the light emitted by the sanitizing devices disclosed herein may be ultraviolet light within the range of far-UVC light and, in some embodiments emits light of, for example, a wavelength of 222 nm, 205 nm, 218 nm, or within a range of approximately 200-240 nm, or within 200-280 nm. Additionally, or alternatively, light emitted by the sanitizing devices disclosed herein may be of 405 nm and/or within a range of 395-430 nm.

Also disclosed herein are so called “smart” or Internet-enabled sanitizing device systems that may include one or more sanitizing devices that are communicatively coupled to one another, a server, one or more sensors, and/or one or more software application running on a computer device operated by a user. This connectivity may enable the user to remotely control and/or monitor the operation of one or more sanitizing devices and/or systems including one or more sanitizing devices.

Also disclosed herein are systems that include one or more sanitizing devices along with other equipment/devices such as sensors, thermometers, thermal cameras, monitors, video cameras, microphones, and/or light reflecting or absorbing materials to determine, for example, how much sanitizing light is needed to adequately sanitize air, surfaces, and/or objects within an environment and/or monitor, for example, how much sanitizing light is incident on a particular region of an environment and/or how many pathogens may be in the air in a particular environment. In some instances, operation of the sanitizing device(s) may be controlled responsively to measurements made by the sensors and/or instructions from a user and/or controller communicated to the sanitizing device(s) via, for example, a server or command communicated to the respective sanitizing device(s) via a communication network such as the Internet.

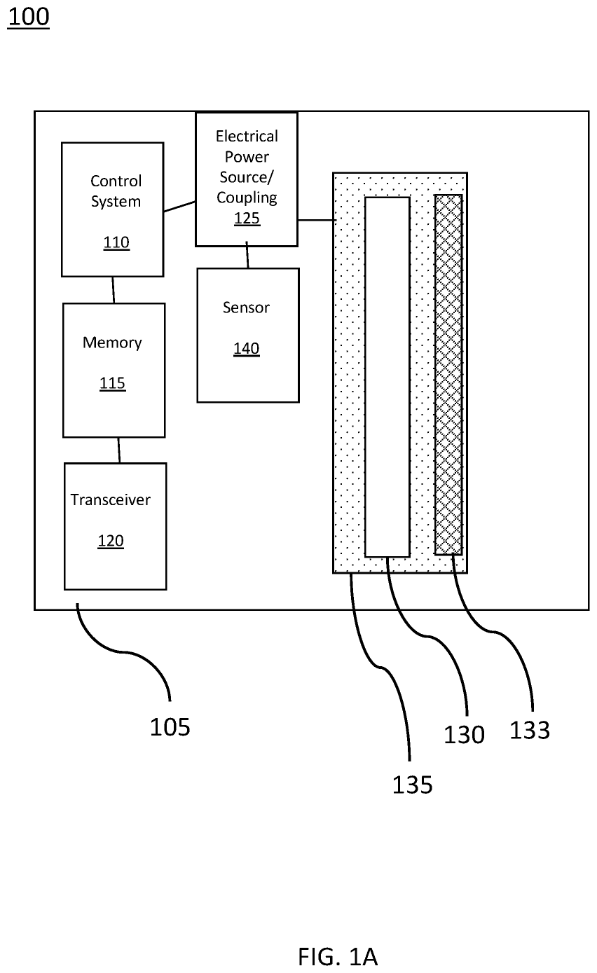

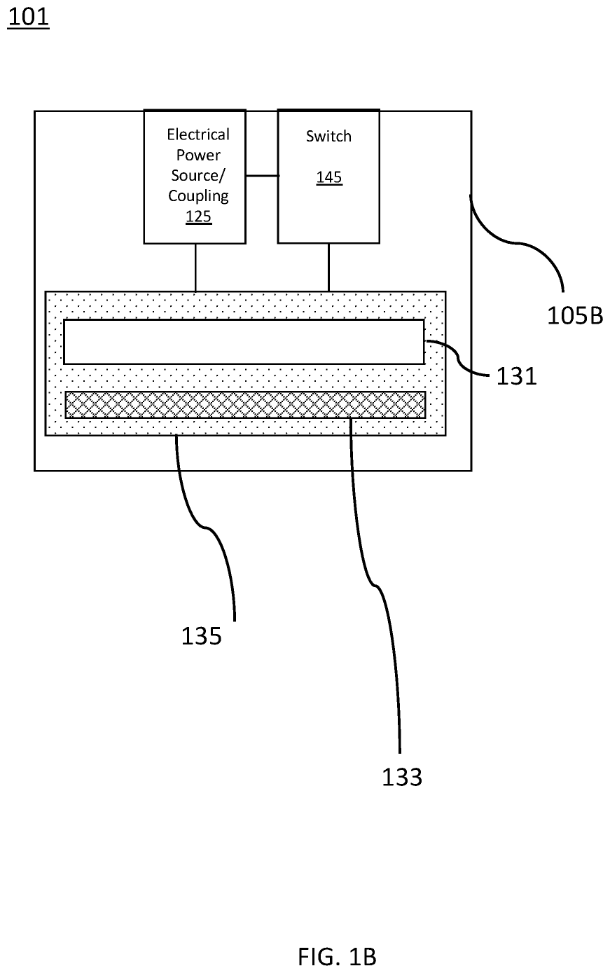

FIG. 1A is a block diagram of a first exemplary first sanitizing device 100 according to the present invention. First sanitizing device 100 includes a housing 105A, a control system 110, a memory 115, a transceiver 120, an electrical power source/coupling 125, a sanitizing light source 130, an optional filtering device 135, an optional reflector 133, and one or more optional sensor(s) 140. Housing 105A may be configured to house the components of first sanitizing device 100 and may be made of any appropriate material or combination of materials including, but not limited to, plastic and metal. Housing 105A may include one or more ports (not shown) by which a power cord may be coupled to first sanitizing device 100 to provide power to first sanitizing device 100 via electrical power source/coupling 125. In some embodiments, electrical power source/coupling 125 may be a battery that, in some cases, may be rechargeable. Additionally, or alternatively, housing 105A may include one or more ports (not shown) by which communication with sanitization device may be facilitated. These ports may be, for example, USB, fire wire, or ethernet ports.

Control system 110 may be configured to control the operation (e.g., intensity of sanitizing light emitted, activate, de-activate, logging the on/off times, control, monitor, and/or track the length of operation of the first sanitizing device 100, etc.) of first sanitizing device 100, sanitizing light source 130, reflector 133, and/or filtering device 135 according to, for example, one or more methods described herein. In some cases, control system 110 may be and/or include a processor and/or a processor/memory combination. Additionally, control system 110 may be configured to receive and/or transmit information from/to transceiver 120 thereby enabling communication of information to and/or from first sanitizing device 100 to an external device or network such as a mesh network, a cloud computing network, and/or the Internet. Transceiver 120 may be configured to receive and/or communicate information via any appropriate communication protocol and/or method including, but not limited to, BLUETOOTH™ and/or Wi-Fi. In some instances, first sanitizing device 100 may be communicatively coupled to a mesh network of devices, sensors, and/or hubs that can connect directly to local phones or other devices for information or connect with the Internet and/or a cloud computing infrastructure so that, for example, the first sanitizing device 100 may operate with/without the Internet.

In some embodiments, control system 110 may be configured to control the intensity of light emitted by sanitizing light source 130, power consumption of first sanitizing device 100 and/or sanitizing light source 130, and/or target radiant flux output of sanitizing light source 130 and/or first sanitizing device 100.

Instructions for operating control system 110 and/or transceiver 120 may be stored on memory 115. Additionally, or alternatively, data acquired by and/or data regarding the operation of first sanitizing device 100 may be stored in memory 110. For example, memory 110 may store, for example, scheduling information, logs of when the first sanitizing device 100 is powered on and off, run times for the first sanitizing device 100, and information from one or more sensors communicatively coupled to first sanitizing device 100.

In some embodiments, first sanitizing device 100 may be Internet enabled and a first sanitizing device 100 may communicate with other sanitizing devices 100 and/or a processor (e.g., a computer, a cloud computing platform, a user's mobile phone, and/or a command center) to receive instructions and/or provide data regarding operation of the first sanitizing device 100. For example, at times, certain operations of the first sanitizing device 100 may be triggered by information received via the Internet such as weather, sunrise/sunset times, a number of mobile phones present in a particular area, and/or road traffic conditions. Additionally, or alternatively, a system (e.g., a reservation or ticketing system) communicatively coupled to first sanitizing device 100 may provide an indication of how many people may be in a particular space at a particular time (e.g., how many people are attending a concert or are scheduled to be present in a classroom), which may be used by the first sanitizing device 100 and/or a processor providing instructions to the first sanitizing device 100 to operate (e.g., turn on/off, set duration of time of operation, etc.) the first sanitizing device 100.

Sanitizing light source 130 may be configured to emit light in the far-UVC portion of the electromagnetic spectrum (i.e., 200-240 nm) and, in some cases, may emit light of 222 nm in wavelength and/or light within a range of approximately 200-240 nm in wavelength. Exemplary sanitizing light sources 130 include, but are not limited to, excimer lamps, micro-plasma lamps, microcavity lamps, UV lamps, UV LEDs, gas discharge lamps, and other gas-excitation-based lamp technologies. In some cases, sanitizing light source 130 may operate cooperatively with filtering device 135 and/or one or more reflector(s) 133 to achieve emission of light that is 222 nm or light within the range of 200-240 nm and/or the focusing or direction of light emitted by the sanitation device in a particular direction or set of directions. Exemplary filtering devices 135 include, but are not limited to, semi-opaque filters, polarizers, and/or mechanical filters (e.g., slits or orifices in the filtering media of filtering device 135). Exemplary optional reflectors 133 include, for example, mirrors or surfaces configured to reflect some, or all, of the light emitted by sanitizing light source 130 in one or more directions so that the light emitted by sanitizing light source 130 may be focused and/or diffused upon exiting sanitization device 100. In some embodiments, reflector 133 is positioned behind sanitizing light source 130 so that it reflects light generated by sanitizing light source 130 away from an inside of first sanitizing device 100 and/or toward the outside of first sanitizing device 100 (e.g., through an aperture in first sanitizing device 100) so that, for example, sanitizing light may be directed toward air, objects, and/or surfaces external to sanitizing device 100.

First sanitizing device 100 may include one or more sensors 140 that may provide information to control system 110 that may cause control system 110 to perform an operation of first sanitizing device 100 such as turning first sanitizing device 100 on or off and/or setting a time duration for the operation of first sanitizing device 100. Additionally, or alternatively, one or more sensors 140 may provide information to a transceiver like transceiver 120 for communication to an external device such as a server, a monitor, and/or a computing device operated by a user.

Exemplary sensors 140 include, but are not limited to, motion sensors, people and/or head counters, thermometers, heat sensors, sensors to detect pathogens or contaminants, carbon dioxide sensors, video cameras, thermal cameras, microphones, sound/noise sensors, temperature sensors, light monitoring sensors configured to monitor an intensity and/or wavelength of light emitted by sanitizing device 100, and/or proximity sensors. Sensor(s) 140 may operate to provide an indication that a person, or group of people, is in an environment in which a sanitizing device is installed and/or operational so that operation of the first sanitizing device 100 may be initiated to sanitize the air and/or objects or surfaces in the environment. In some embodiments, one or more sensors 140 may be a color sensor. The color sensor may operate as a color UV exposure strip.

In some embodiments, first sanitizing device 100 may be a component of a sanitizing system that includes, for example, a plurality of sanitizing devices 100/101 and/or devices to assist with the disbursement of the sanitizing light around an environment or room such as reflectors, mirrors, lenses, light absorption materials (e.g., fabric, foam, opaque lenses, and/or semi-transparent lenses) and/or light reflecting materials such as reflectors or glass. In some instances, for example, a room may include a mirror positioned at each corner of the room to redirect the sanitizing light back into the room and along the walls of a room so that the walls are sanitized. Additionally, or alternatively, reflective devices may be positioned within a room so that air and/or objects positioned below a surface or object (e.g., a table or countertop) are exposed to the sanitizing light when that light is initially projected from above. Additionally, or alternatively, a sanitizing system may include a sanitizing light absorption material positioned in areas of an environment or room where the sanitizing light may be too concentrated and/or to prevent the sanitizing light from reaching a place it is undesired such as outside the room.

In some embodiments, the sanitizing system may include one or more air circulation devices such as fans, to circulate air within a room so that the air in the room is evenly exposed to the sanitizing light from first sanitizing device 100. In some cases, the air circulation devices may be communicatively coupled via, for example, the Internet and/or a mesh network so that they are activated when the first sanitizing device 100 is activated.

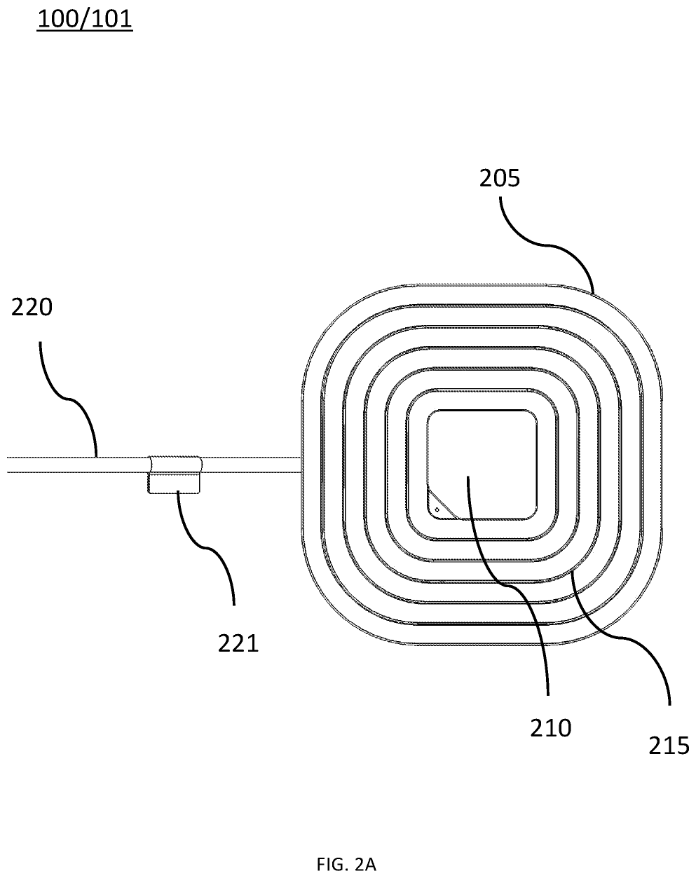

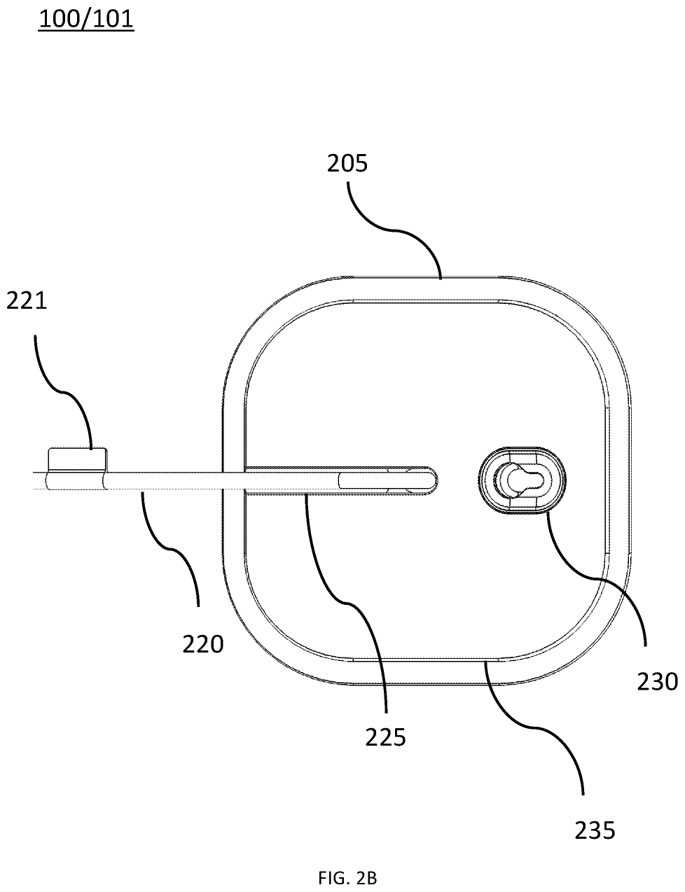

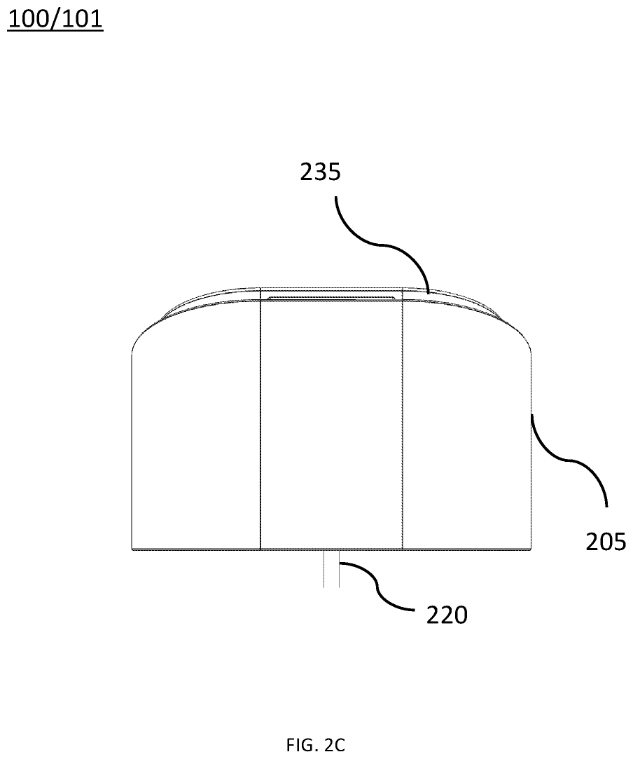

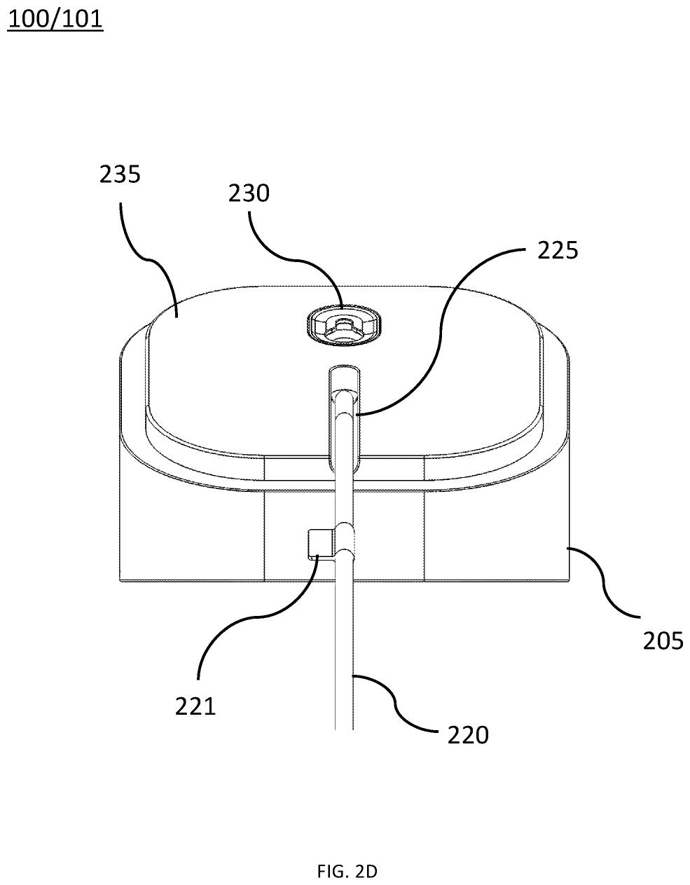

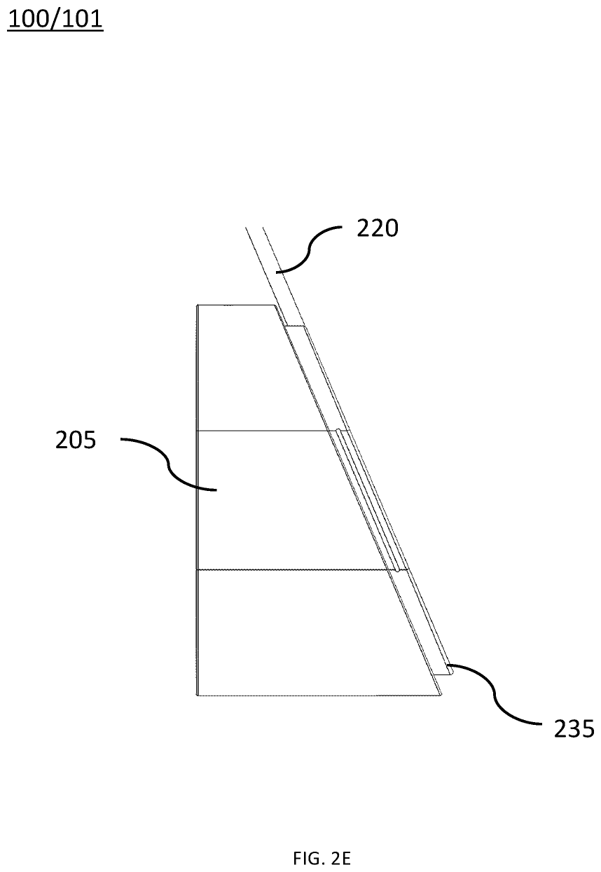

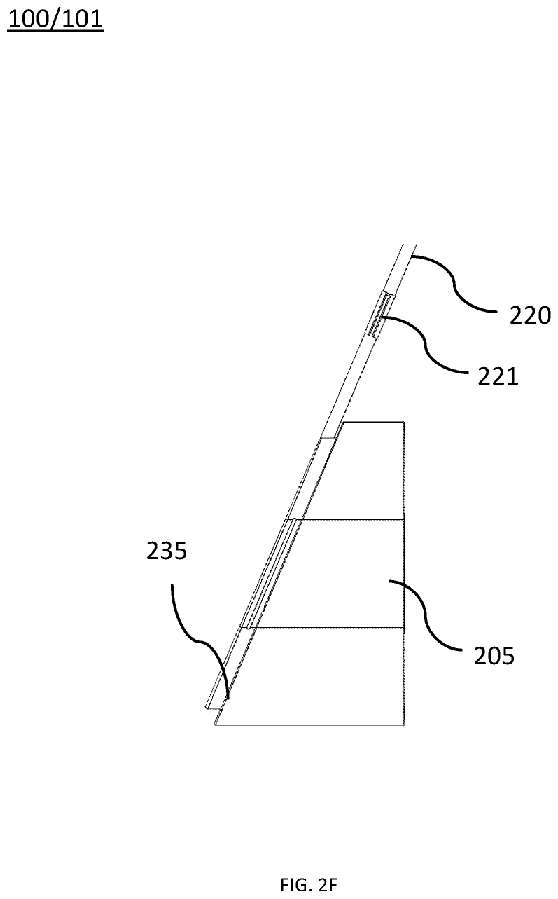

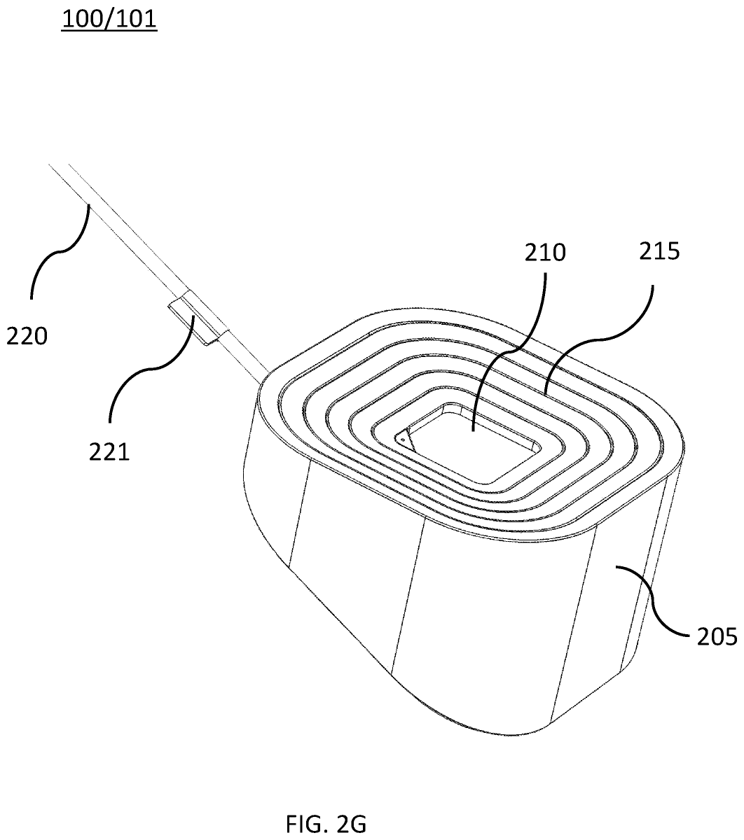

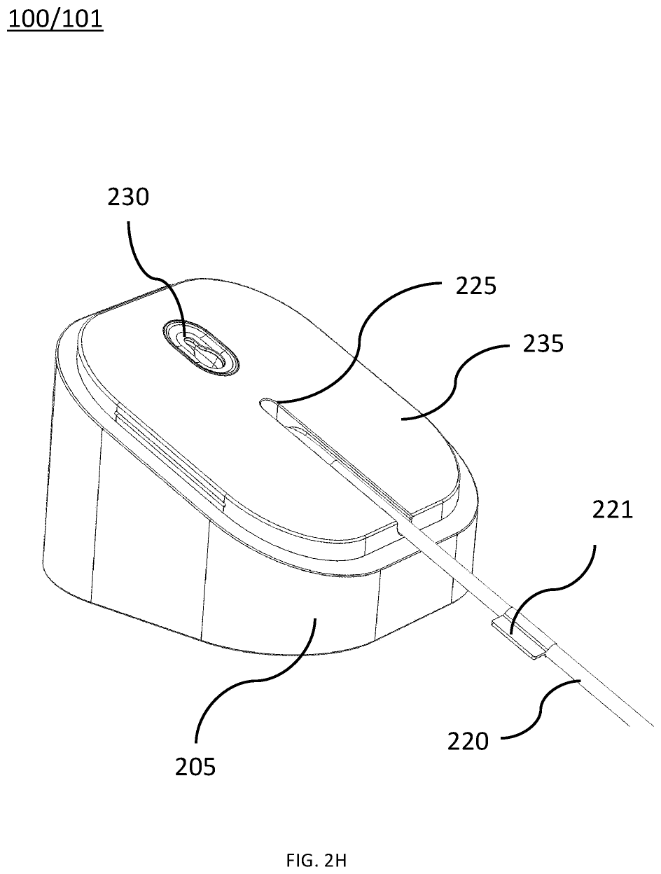

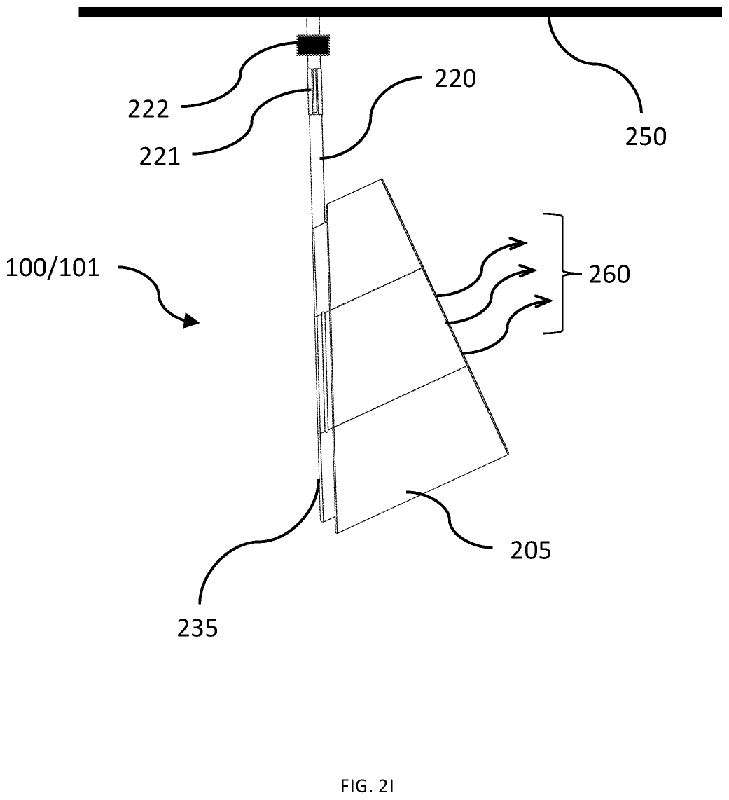

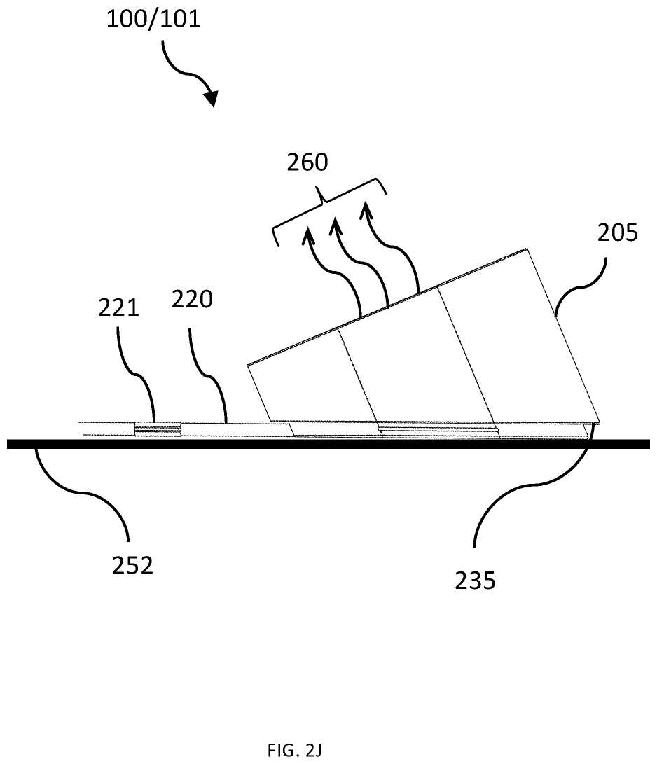

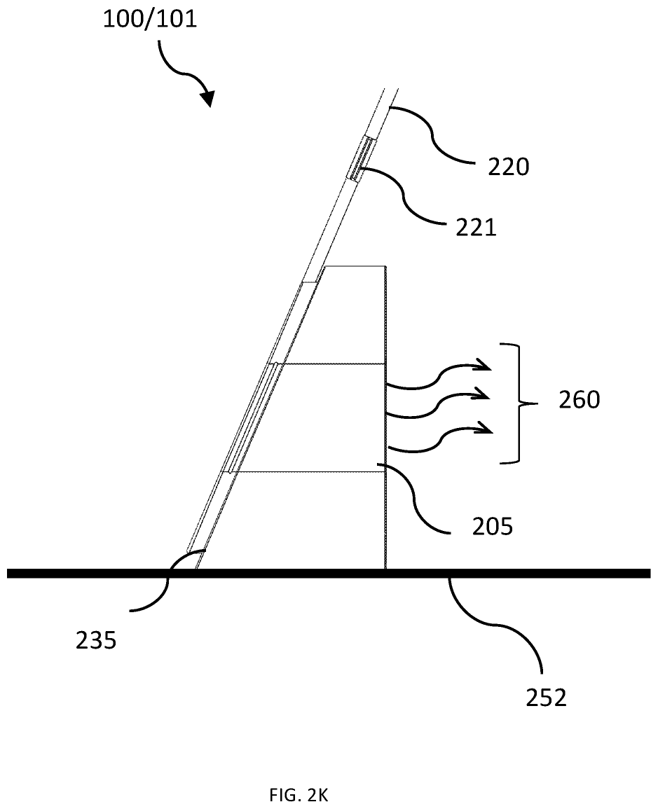

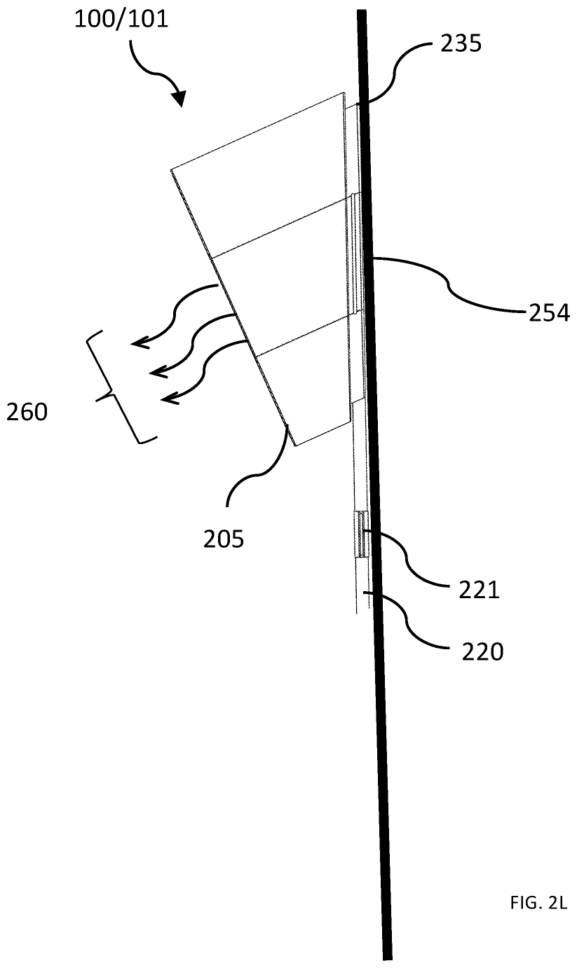

In some embodiments, a first sanitizing device 100 and/or a housing 105A may be configured to move and/or rotate in order to, for example, project sanitizing light around an environment or room. This movement and/or rotation may be facilitated by, for example, a movement mechanism 222 as shown in FIG. 2I and discussed below. Alternatively, first sanitizing device 100 and/or a housing 105A may be configured to be ceiling hung, wall mounted, and/or positioned on a surface such as a table or counter.

At times, first sanitizing device 100 may be mounted so that it is a preferred distance from a surface and/or object. The preferred distance may be determined by determining a concentration, or intensity, of sanitizing light that has to reach a surface of the object to sanitize it. For example, it may be determined that a sanitizing device must be less than 3, 6, 8, or 15 feet above a surface of an object to sanitize the surface of the object. In this example, first sanitizing device 100 may be positioned in an environment or room with ceilings below 15 feet in height or be suspended from higher ceilings so the first sanitizing device 100 is within 15 feet from the surface of objects within the room.

FIG. 1B is a block diagram of a second exemplary sanitizing device 101 according to the present invention. Sanitizing device 101 includes a housing 105B, electrical power source/coupling 125, a switch 145, a sanitizing light source 131, an optional reflector 133, and optional filtering device 135 and is configured to emit light that have a wavelength of 222 nm or approximately 222 nm. Housing 105B may be configured to house the components of sanitizing device 101 and may resemble housing 105A. Housing 105B may include one or more ports (not shown) by which a power cord may be coupled to first sanitizing device 100 to provide power to first sanitizing device 100 via electrical power source/coupling 125. In some embodiments, electrical power source/coupling 125 may be a battery that, in some cases, may be rechargeable. Second sanitizing device 101 may be activated via switch 145 which may be, for example, a manual and/or electronic switch that turns sanitizing light source 131 on and off. Sanitizing light source 131 may be similar to sanitizing light source 130 but it may be specifically configured to only emit light that is 222 nm in wavelength. Additionally, or alternatively, light emitted by sanitizing light source 131 may be filtered using optional filtering device 135 so that the wavelength of the light emitted by second exemplary sanitizing device 101 is of a preferred wavelength that sanitizes surfaces, air, and/or objects but does not harm human tissue such as light of 222 nm, 405 nm, and/or within a range of 210-280, 200-240 nm, and/or 390-420 nm. Exemplary optional reflectors 133 include, for example, mirrors or surfaces configured to reflect some, or all, of the light emitted by sanitizing light source 130 in one or more directions so that the light emitted by sanitizing light source 130 may be focused and/or diffused upon exiting sanitization device 100.

FIGS. 2A-2H provide different views of an exemplary sanitizing devices 100 and/or 101 (which may be collectively referred to herein as “sanitizing devices 100/101”). More particularly, FIG. 2A is a top plan view of an exemplary embodiment of sanitizing device 100/101; FIG. 2B is a bottom plan view of an exemplary embodiment of sanitizing device 100/101; FIG. 2C is a front plan view of an exemplary embodiment of sanitizing device 100/101; FIG. 2D is a back plan view of an exemplary embodiment of sanitizing device 100/101; FIG. 2E is a first-side plan view of an exemplary embodiment of sanitizing device 100/101; FIG. 2F is a second-side plan view of an exemplary embodiment of sanitizing device 100/101; FIG. 2G is a top perspective view of an exemplary embodiment of sanitizing device 100/101; and FIG. 2H is a bottom perspective view of an exemplary embodiment of sanitizing device 100/101. An overall shape of the sanitizing devices 100/101 shown in FIGS. 2A-2H is that of a truncated rectangular prism with a square horizontal cross section. The corners of the square cross section are rounded along the height of the sanitizing devices 100/101. Exemplary horizontal cross-sectional dimensions are 2-10 inches in length and width. An exemplary height of sanitizing devices 100/101 is 1-20 inches in height for the front side (as shown in FIG. 2C) and an exemplary height of sanitizing devices 100/101 is 0.5-10 inches in height for the back side (as shown in FIG. 2D). However, in most embodiments, a height of a front of sanitizing device 100/101 is 25-75% larger than a height of the back of sanitizing device 100/101. The first- and second-side views of FIGS. 2E and 2F show sanitizing device 100/101 oriented so that the front of sanitizing device 100/101 is toward the bottom of the page and show exemplary relative proportions for the front and back of housing 205 that contribute to the housing's truncated rectangular prism shape.

Sanitizing device 100/101 includes a housing 205 that defines the size and shape of sanitizing device 100/101 and also houses all of the components (with the exception of entire length of the power cord) included in sanitizing device 100/101. Sanitizing device 100/101 also includes a covering 210 for sanitizing light source 130. In some embodiments, covering 210 may be configured to filter light emitted by sanitizing light source 130 so that only light of a desired wavelength, or range of wavelengths, is emitted by sanitizing device 100/101. Covering 210 may be any color, or a combination of colors.

A facing 215 of housing 205 may be configured to have optional step-down levels wherein covering 210 is positioned below (e.g., 0.25-2 inches) an upper edge of facing 215 so that an upper surface of sanitizing device 100/101 has a three-dimensional shape that indents into the upper surface. In the embodiment shown in the figures, facing 215 has six (6) optional steps that progressively go deeper into sanitizing device 100/101 until reaching covering 210. In some cases, the step-like upper surface of sanitizing device 100/101 may be configured to amplify and/or direct light exiting from cover 215. Sanitizing device 100/101 also includes a power and/or communication cord 220, an end of which may be configured to electrically couple to an outlet and/or electrically/communicatively couple to an adaptor (e.g., USB, USB-C, etc.). Power/communication cord 220 may be coupled to a clip 221 configured to clip to another portion of power/communication cord 220 and/or another object so that, for example, power/communication cord 220 may be neatly stored.

Housing 205 may also include a notch 230 or opening by which to wall-mount sanitizing device 100/101 to a wall or other surface as shown in FIG. 2B. Notch may be configured to allow an object, like a nail or screw head to be inserted into a circular portion of notch 230 and then slide down into a groove so as to be securely attached to the object projecting from the wall (i.e., the nail or screw). FIG. 2B also shows a channel 225 in which a portion of power/communication cord 220 may reside so that, for example, a back surface of sanitizing device 100/101 may sit flatly on a surface (e.g., a horizontal or vertical surface). Housing 205 may have a raised portion 235 that projects outward from the back surface of sanitizing device 100/101. Notch 230 and/or channel 225 may be recessed into raised portion 235

FIGS. 2I-2L provide side plan views of the sanitizing device 100/101 of FIGS. 2A-2H arranged in different orientations in an environment and/or on a surface of an object. More specifically, FIG. 2I provides a side view of sanitizing device 100/101 suspended from a ceiling 250 via suspension/power/communication cord 220. FIG. 2I also shows exemplary sanitizing light (in the form of a set of wavy lines 260) being projected from the covering 210 of housing 205. In the embodiment of FIG. 2I, a movement mechanism 222 is coupled to suspension/power/communication cord 220. Movement mechanism 222 may be configured to move sanitizing device 100/101 up and down, change an orientation of sanitizing device 100/101, and/or rotate sanitizing device 100/101 by 360 degrees or a portion thereof. Exemplary movement mechanisms 222 include, but are not limited to, motors, stepper motors, a winch, and/or a pulley and cord system.

FIG. 2J shows sanitizing device 100/101 oriented so that the back of the sanitizing device is resting on a horizontally-oriented surface such as a table or countertop. FIG. 2J also shows exemplary sanitizing light (in the form of a set of wavy lines 260) is projected at an upward angle from the covering 210 of housing 205.

FIG. 2K shows sanitizing device 100/101 oriented so that the front of the sanitizing device is resting on a horizontally-oriented surface such as a table or countertop so that sanitizing light (shown as set of wavy lines 260) is projected from the covering 210 of housing 205 in a substantially horizontal direction.

FIG. 2L shows sanitizing device 100/101 mounted to a wall 254 via notch 230 so that the back of the sanitizing device coincident with wall 254 and sanitizing light (shown as set of wavy lines 260) is projected from the covering 210 of housing 205 in at a downward angle.

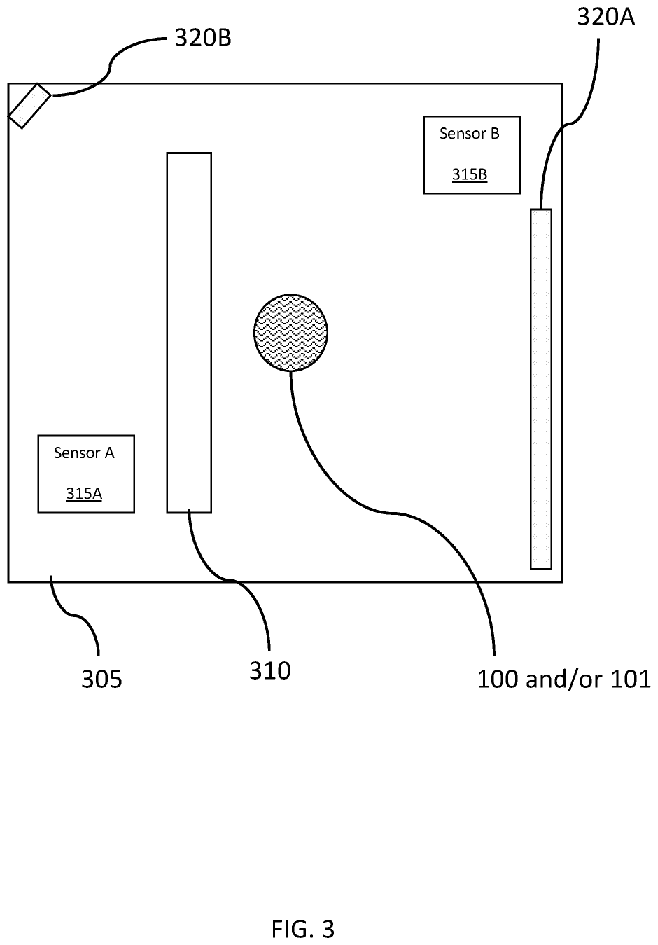

FIG. 3 is a diagram of an exemplary environment 305 in which first and/or second sanitizing device 100 and/or 101 and/or a sanitizing system may operate to sanitize, or disinfect, surfaces, air, and/or objects included therein. Environment 305 may be, for example, a retail outlet, store, restaurant, food preparation areas/station, transportation hub, public space, bathroom, hotel, school, gym, medical/dental office, airport lounge, and hotel room. Environment 305 may be defined by one or more walls. Environment 305 includes first and/or second sanitizing device 100 and/or 101, an object 310, a first sensor 315A, a second sensor 315B, a first reflective object 320A, and a second reflective object 320B. Although only two sensors 315 and reflective objects 320 are depicted in FIG. 3, it will be understood that any number of sensors 315 and/or reflective objects 320 may be used in environment 305.

First and second sensors 315A and 315B may be sensors configured to monitor environment 305 for the presence of, for example, people or pathogens. First sensor 315A and/or second sensor 315B may be, for example, a motion sensor, a heat sensor, a sensor to detect pathogens or contaminants, a people counter, a head counter, a temperature sensor, a heat camera, a carbon dioxide sensor, a sound/noise sensor, a temperature sensor, a light intensity sensor, a light color sensor, and/or a proximity sensor.

External sensors 315 and/or internal sensor 140 may be configured share some, or all, of the same capabilities and/or operations. For example, in one embodiment, a sensor 315 and/or 140 may be temperature sensor and/or heat camera configured to measure a body temperature of people within, entering, and/or exiting an environment. This information may be conveyed to, for example, a sanitizing device and/or a server communicatively coupled to the server so that, for example, an operation of a sanitizing device 100/101 may be responsive thereto. For example, if it is determined that an individual with an elevated body temperature has entered an environment, sanitizing device 100/101 may be activated to neutralize any pathogens the individual may be shedding or breathing into the environment.

Additionally, or alternatively, sensor 315 and/or 140 may be a light meter that measures an intensity of light (in, for example, kilojoules) in a particular portion of the environment to see whether, for example, sanitizing device 100/101 is working properly and/or whether the particular portion of the environment is receiving enough/too much sanitizing light to be effective.

Additionally, or alternatively, sensor 315 may be a photo-responsive material that may, for example, change color in the presence of light of a certain wavelength, set of wavelengths, and/or intensity. This may enable a sensor and/or user to, for example, verify that sanitizing device 100/101 is operating properly and/or an area of the environment corresponding to the photo-sensitive material is receiving sufficient sanitizing light to be effectively sanitized.

In one exemplary implementation, a sanitizing device 100/101 may be placed in a food preparation area so that the area and the food remains sanitized while food is being prepared and/or is waiting to be consumed (e.g., under a heat lamp or on a hot food bar). Additionally, or alternatively, sanitizing device may be used to sanitize an area where raw food such as sushi or salad is being prepared in order to deactivate any food-borne pathogens that may be present on/in the food prior to consumption.

In some instances, information from first sensor 315A and/or second sensor 315B may be communicated to first sanitizing device 100, received by transceiver 120, and used by control system 110 to operate first sanitizing device 100. Additionally, or alternatively, information from first sensor 315A and/or second sensor 315B may be communicated to a processor not resident within first sanitizing device 100 such as a processor operating on a cloud computing platform communicatively coupled to first sensor 315A, second sensor 315B, and/first sanitizing device 100 via, for example, the Internet and/or a mesh network to control the operation of first sanitizing device 100.

Object 310 may be, for example, a table or a counter on which food is eaten or prepared. Alternatively, object 310 may be a counter over which people exchange information or transact business that may be used in, for example, a hotel or governmental agency. First and/or second sanitizing device 100 and/or 101 may be arranged in environment 305 in a position that facilitates delivery of a sufficient amount (e.g., intensity) of sanitizing light to the surface of object 310 to sanitize the surface of object 310.

In some embodiments, first and/or second sensors 315A and/or 315B may be arranged in portions of environment 305 to monitor the effectiveness of first and/or second sanitizing device 100 and/or 101. For example, in some instances, a sensor 315 may be placed in a dead spot in environment 305 or placed where verification that the device is working is of particular interest such as under object 310 (not shown). Information from first and/or second sensors 315A and/or 315B may be communicated to, for example, sanitizing device 100 and/or a processor communicatively coupled to first and/or second sensors 315A and/or 315B via, for example, the Internet.

In some embodiments, first sanitizing device 100, first and/or second sensors 315A and/or 315B may be configured to generate a report indicating, for example, when a sanitizing device is on, a duration of time the sanitizing device is operating, a sensor readout (e.g., carbon dioxide levels in the environment, when the lights are turned on, how often a motion sensor is activated, etc.). In some cases, these reports may be communicated to a remote entity via a network such as the Internet.

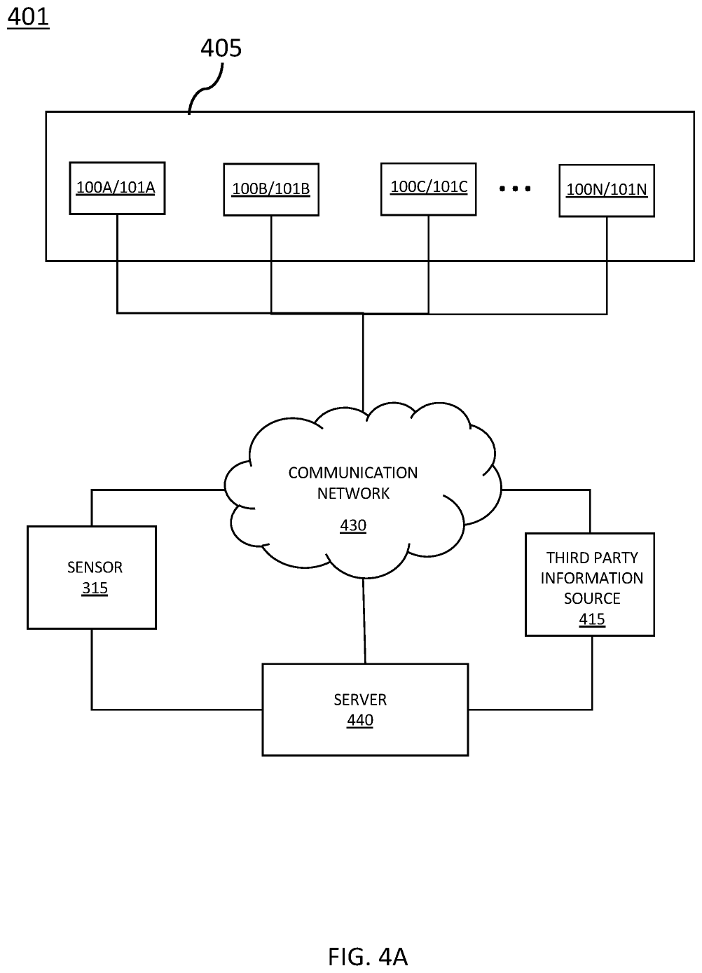

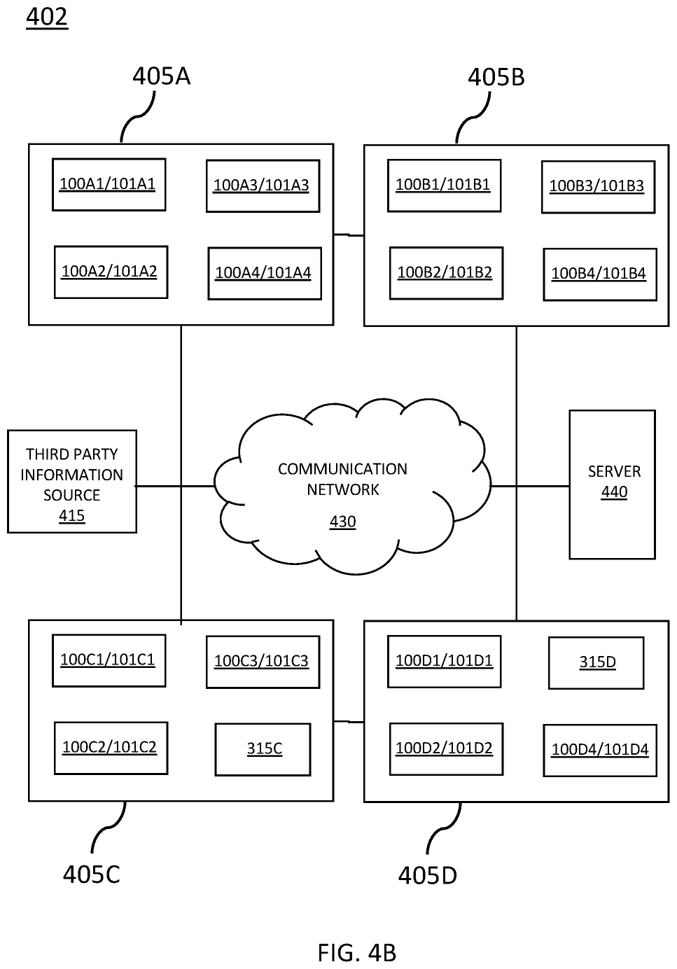

FIG. 4A is a diagram of a first exemplary system 301 deploying a set 405 of sanitizing devices 100 and/or 100 communicatively connected to on another via a communication network such as the Internet, a mesh network, a private network, and/or a local area network (LAN). Set 405 may have any number (e.g., 2, −500) sanitizing devices 100 and/or 101 that may be arranged in any configuration. For example, all sanitizing devices 100 and/or 101 in a set may reside in an environment (e.g., room or region of a room), a building, or a set of buildings. In another example, sanitizing devices 100 and/or 101 in a set may be geographically disperse from one another (e.g., spread out over a housing development, strip mall, set of businesses operating under unified management, and/or a set of users who are subscribers to a service for monitoring and/or controlling the use sanitizing devices 100/101 within the respective environments for the sanitizing devices 100/101).

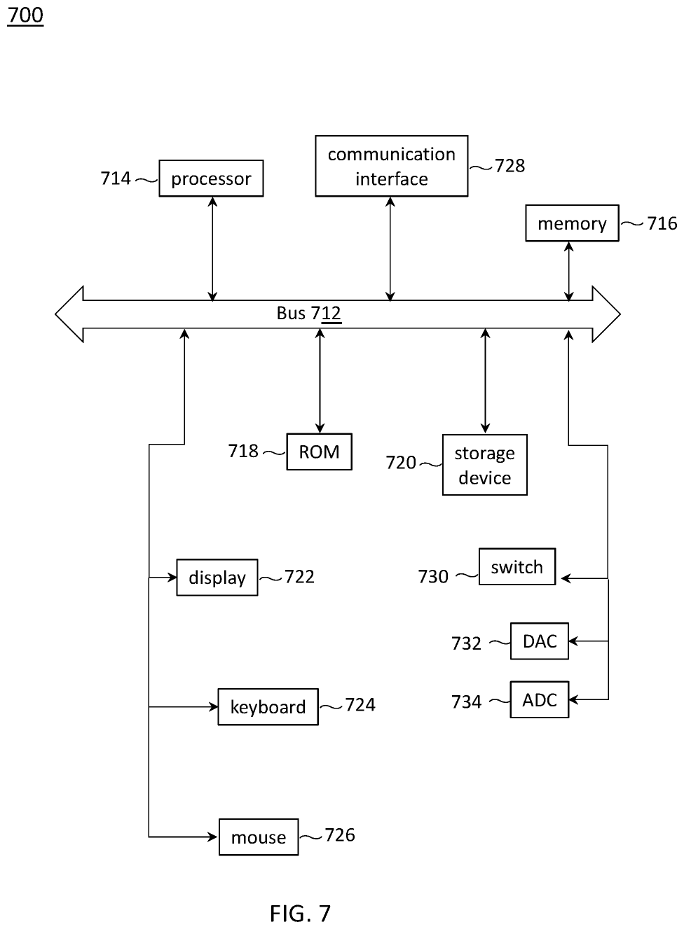

In the example of FIG. 4A, set 405 include sanitizing devices 100A/101A, sanitizing devices 100B/101B, sanitizing devices 100C/101C through to sanitizing devices 100N/101N. Set 405 and/or individual sanitizing devices 100A/101A, sanitizing devices 100B/101B, sanitizing devices 100C/101C through to sanitizing devices 100N/101N may be communicatively coupled to one or more sensors 315, a server 440, and/or a third party information source 415 via communication network 430. One or more sensors 315 may be configured to, for example, monitor one or more aspect of an environment in which sanitizing devices 100A/101A, sanitizing devices 100B/101B, sanitizing devices 100C/101C through to sanitizing devices 100N/101N are placed. Server 440 may be configured (via, for example, a set of instructions stored thereon) to monitor and/or control operations for sanitizing devices 100A/101A, sanitizing devices 100B/101B, sanitizing devices 100C/101C through to sanitizing devices 100N/101N according to, for example, one or more methods disclosed herein. The monitoring of sanitizing devices 100A/101A, sanitizing devices 100B/101B, sanitizing devices 100C/101C through to sanitizing devices 100N/101N may include, but is not limited to, determining when sanitizing devices 100A/101A, sanitizing devices 100B/101B, sanitizing devices 100C/101C through to sanitizing devices 100N/101N, respectively, are on or off, determining what, if any, data sanitizing devices 100A/101A, sanitizing devices 100B/101B, sanitizing devices 100C/101C through to sanitizing devices 100N/101N, respectively, are receiving from one or more sensors like sensor 315, controlling a position and/or orientation of sanitizing devices 100A/101A, sanitizing devices 100B/101B, sanitizing devices 100C/101C through to sanitizing devices 100N/101N, respectively, and/or monitoring sanitizing devices 100A/101A, sanitizing devices 100B/101B, sanitizing devices 100C/101C through to sanitizing devices 100N/101N, respectively, for one or more error conditions. In some embodiments, server 440 may be configured to be operated by a user of system 400 via one or more control panels or computer user interfaces that may be displayed on a display device such as display device 722 discussed below with regard to FIG. 7.

Third party information source 415 may be any source of information that may provide information that is helpful to the monitoring and/or controlling of one or more sanitizing devices 100A/101A, sanitizing devices 100B/101B, sanitizing devices 100C/101C through to sanitizing devices 100N/101N included in within set 405. Exemplary third party information sources include websites, subscription databases, governmental information sources, and/or databases. In one example, a third party information source may be a governmental agency in charge of population health data (e.g., the Centers for Disease Control (CDC)) and server 440 may query third party information source 415 for information pertaining to disease infection rates, which may be geographically specific. Server 440 may then use this information to control the operation of one or more sanitizing devices 100A/101A, sanitizing devices 100B/101B, sanitizing devices 100C/101C through to sanitizing devices 100N/1. For example, if sanitizing devices 100A/101A and sanitizing devices 100B/101B are positioned within a county with relatively high disease infection rates, then server may responsively control an operation of sanitizing devices 100A/101A and/or sanitizing devices 100B/101B so that they operate more frequently and/or for a longer time in order to sanitize environments in that county to mitigate the risks associated with a higher rate of infection in the county. In another example, if all the sanitizing devices 100/101 included within set 405 are positioned in a city, then server 440 may query third party information source for information regarding the average and/or high temperature in the city so that operation of one or more of the sanitizing devices 100A/101A, sanitizing devices 100B/101B, sanitizing devices 100C/101C through to sanitizing devices 100N/101N may be adjusted accordingly (e.g., more frequent operation when average/high temperatures are conducive to bacteria/pathogen growth and less frequent operation when average/high temperatures are not conducive to bacteria/pathogen growth).

FIG. 4B is a diagram of a second exemplary system 402 including a plurality of sets of communicatively connected sanitizing devices 405A, 405B, 405C, and 405D that are communicatively coupled to server 440 and/or third party information source 415 via communication network 430 and/or wired communication links. More specifically, system 402 includes a first set of four sanitizing devices 100A1/101A1, 100A2/101A2, 100A3/101A3, and 100A4/101A4; a second set 405B of four sanitizing devices 100B1/101B1, 100B2/101B2, 100B3/101B3, and 100B4/101B4; a third set 405C of three sanitizing devices 100C1/101C1, 100C2/101C2, 100C3/101C3, and a sensor 315C; and a fourth set 405D of three sanitizing devices 100D1/101D1, 100D2/101D2, 100D3/101D3, and a sensor 315D. It will be understood by those of skill in the art that system 402 may have any number (e.g., 2-1000) sets of communicatively connected sanitizing devices 405.

System 402 may operate in a manner similar to system 401 but on a larger scale with a plurality of four sets of sanitizing devices. In the example of FIG. 4B, four sets of sanitizing devices are shown and, in some cases, a sensor 315 may be included in a set 405 as is the case with third and fourth sets 405C and 405D. Sensor 315 positioned within a set 405C and/or 405D may monitor one or more environmental conditions and/or perform operations that may be communicated to, for example, sanitizing devices included in set 405C or 405D, respectively, first and/or second set 405A and/or 405B, and/or server 440.

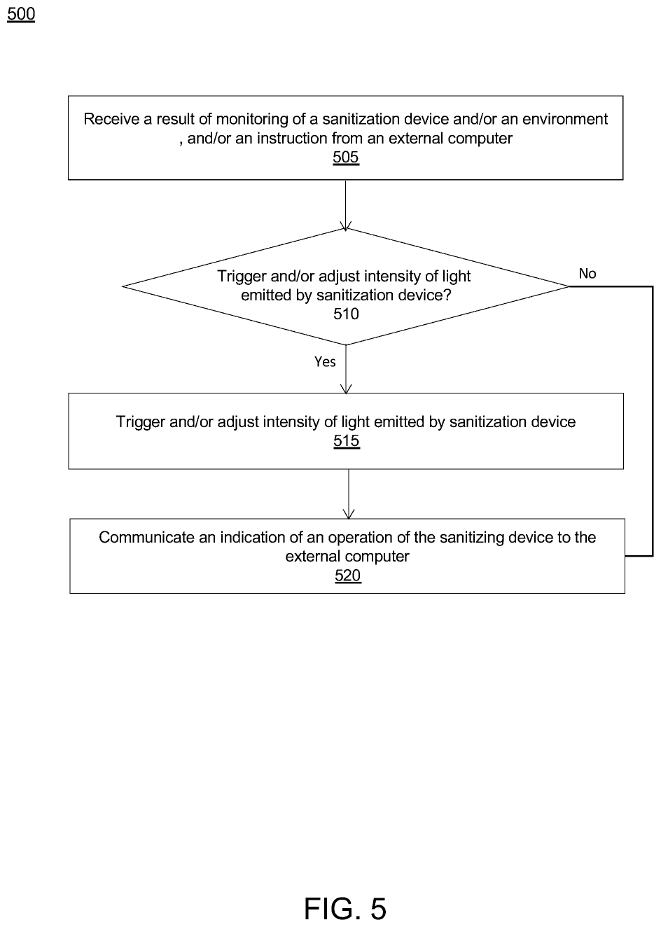

FIG. 5 is a flowchart illustrating a process 500 for operating a sanitizing device like sanitizing device 100 and/or 101. Process 500 may be executed by, for example, sanitizing device 100 and/or 101, system 401, system 402, and/or components thereof.

In step 505, a result of monitoring of the sanitizing device and/or an environment and/or an instruction from an external computer, such as server 440, may be received by a sanitizing device such as sanitizing device 100 and/or 101. In some embodiments, the monitoring result may be received from a sensor resident within a housing for the sanitizing device, such as sensor 140. Additionally, or alternatively, the monitoring result may be received from an external monitor, such as monitor 315 shown in FIGS. 3, 4A, and 4B and discussed above. Exemplary monitoring results include, but are not limited to, a change in an ambient temperature, a sensed motion, a lack of sensed motion, and/or an indication of air motion within an environment. In some embodiments, the monitoring result may be from a temperature sensor that monitors a temperature of the sanitizing device so that it may be automatically turned off by, for example, a switch such as switch 145, a controller and/or external device such as server 440 in the event of overheating.

In step 510, the received monitoring result may be analyzed to determine whether an intensity of light (in most cases light of a wavelength within the far UVC range) emitted by the sanitizing device should be adjusted (e.g., increased or decreased) and/or if an operation of the sanitizing device should be triggered (e.g., should the sanitizing device be turned on or off) and, if so, an operation of the sanitizing device may be triggered and/or adjusted accordingly (step 515). When an operation of the sanitizing device is not triggered and/or adjusted (step 510) or following execution of step 515, an indication of the operation of the sanitizing device (e.g., when the sanitizing device is turned on/emitting far UVC light; when the sanitizing device is off; when an intensity of the sanitizing device is adjusted) may be provided to an external computer (e.g., server 440) and/or a personal computing device (e.g., smart phone or tablet computer) operated by a user via a display device like display device 722.

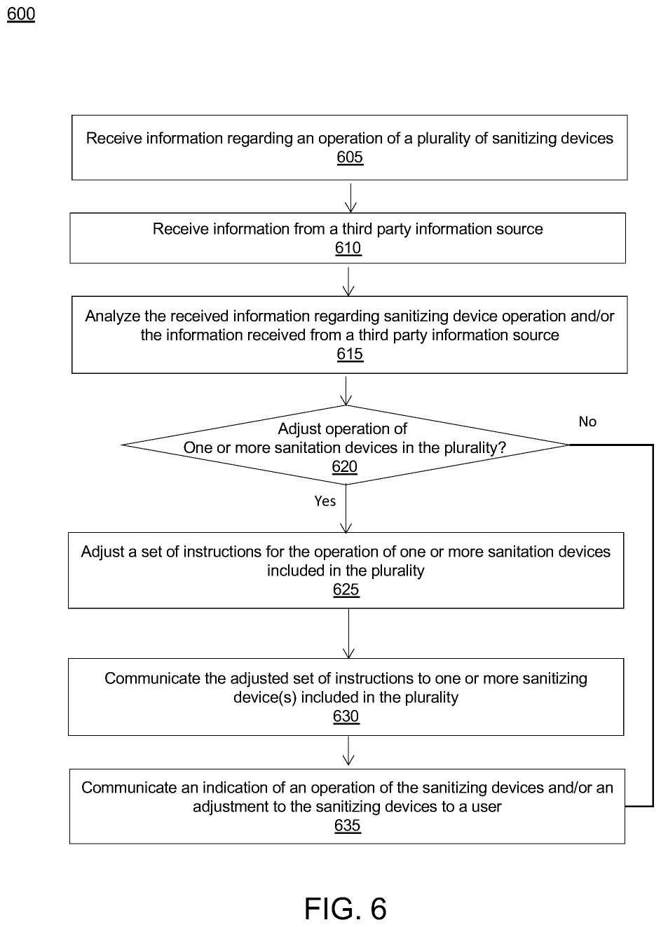

FIG. 6 is a flowchart illustrating a process 600 for centrally operating a plurality and/or set of sanitizing devices such as the sets of sanitizing device included in systems 401 and 402. Process 600 may be executed by and/or in, for example, server 440, system 401, system 402, and/or components thereof.

In step 605, information regarding an operation of one or more sanitizing devices, such as sanitizing device 100/101 and/or a set of sanitizing devices like set 405, 405A, 405B, 405C, and/or 405D may be received. The information that is received may be, for example, information regarding how long

A schedule, of operation and/or intensity adjustments for one or more of the sanitizing devices providing information received in step 605,

Optionally, information from one or more sensors like sensor 140 and/or 215 may also be received in step 605. Exemplary information that may be received from the one or more sensors includes, but is not limited to, air purification rates, a number of times a motion sensor is activated (e.g., motion is detected) in a time period (e.g., every hour, every 12 hours, every 24 hours, etc.), a number of particulates in the ambient air of an environment, a temperature of an environment, and a position of objects within an environment.

Optionally, in step 610, information from a third party information source, such as third party information source 415 may be received. Exemplary third party information includes, but is not limited to, an air quality index (AQI) measurement, a temperature, a level of humidity, and/or an indication of how many individuals within a geographic area are diagnosed with a disease (e.g., the flu, COVID-19, the common cold, etc.), population, or crowd size, counts, audio information about an environment (e.g., conversation noise level, how many times a certain sound (e.g., a tone a machine makes when it is finished executing a process such as processing a credit card or scanning a bar code is heard, etc.) and/or video information that may, for example, track how many people are in a certain space over time.

In step 615, the information received in step 605 and 610 (when received) may be analyzed to determine whether an adjustment in the operation of one or more sanitizing devices included in the plurality of sanitizing devices may be necessary (step 620) and, if so, a set of instructions pertaining to the operation of one or more sanitizing devices included in the plurality of sanitizing devices may be adjusted according to the analysis (step 625) and communicated to the respective one or more sanitizing devices included in the plurality of sanitizing devices (step 630). When an adjustment is not necessary in step 620 and/or following completion of step 630, an indication of an operation of the sanitizing devices and/or an adjustment to the operation of the sanitizing devices may be provided to the user via, for example, a graphic user interface displayed on a display device (step 635).

FIG. 7 provides an example of a processor-based system 700 that may store and/or execute instructions for one or more of the processes described herein. Processor-based system 700 may be resident in a server, like server 440 and/or a sanitizing device like sanitizing device 100 and/or 101. Note, not all of the various processor-based systems which may be employed in accordance with embodiments of the present invention have all of the features of system 700. For example, certain processor-based systems may not include a display inasmuch as the display function may be provided by a client computer communicatively coupled to the processor-based system or a display function may be unnecessary. Such details are not critical to the present invention.

System 700 includes a bus 712 or other communication mechanism for communicating information, and a processor 714 coupled with the bus 712 for processing information. System 700 also includes a main memory 716, such as a random-access memory (RAM) or other dynamic storage device, coupled to the bus 712 for storing information and instructions to be executed by processor 714. Main memory 716 also may be used for storing temporary variables or other intermediate information during execution of instructions to be executed by processor 714. System 700 further includes a read only memory (ROM) 718 or other static storage device coupled to the bus 712 for storing static information and instructions for the processor 714. A storage device 720, which may be one or more of a hard disk, flash memory-based storage medium, a magnetic storage medium, an optical storage medium (e.g., a Blu-ray disk, a digital versatile disk (DVD)-ROM), or any other storage medium from which processor 714 can read, is provided and coupled to the bus 712 for storing information and instructions (e.g., operating systems, applications programs and the like).

System 700 may be coupled via the bus 712 to a display 722, such as a flat panel display, for displaying information to a user. An input device 724, such as a keyboard including alphanumeric and other keys, may be coupled to the bus 712 for communicating information and command selections to the processor 714. Another type of user input device is cursor control device 726, such as a mouse, a trackball, or cursor direction keys for communicating direction information and command selections to processor 714 and for controlling cursor movement on the display 722. Other user interface devices, such as microphones, speakers, etc. are not shown in detail but may be involved with the receipt of user input and/or presentation of output.

The processes referred to herein may be implemented by processor 714 executing appropriate sequences of processor-readable instructions stored in main memory 716. Such instructions may be read into main memory 716 from another processor-readable medium, such as storage device 720, and execution of the sequences of instructions contained in the main memory 716 causes the processor 714 to perform the associated actions. In alternative embodiments, hard-wired circuitry or firmware-controlled processing units (e.g., field programmable gate arrays) may be used in place of or in combination with processor 714 and its associated computer software instructions to implement the invention. The processor-readable instructions may be rendered in any computer language.

System 700 may also include a communication interface 728 coupled to the bus 712. Communication interface 728 may provide a two-way data communication channel with a computer network, which provides connectivity to the plasma processing systems discussed above. For example, communication interface 728 may be a local area network (LAN) card to provide a data communication connection to a compatible LAN, which itself is communicatively coupled to other computer systems. The precise details of such communication paths are not critical to the present invention. What is important is that system 700 can send and receive messages and data through the communication interface 728 and in that way communicate with other controllers, etc.

System 700 may also include one or more switches, dials, or devices 730 that facilitate manual operation (e.g. on/off, intensity of UV light emitted, scheduling of operation, etc.) of one or more sanitization devices like sanitizing device 100 and/or 101. In some embodiments, system 700 may further include one or more digital-to-analog converters (DACs) 732 and/or analog-to-digital converters (ADCs) 734 that may be configured to operate as, for example, input/output modules for data communicated into and/or out of one or more sanitization devices like sanitizing device 100 and/or 101 and/or control one or more operations of one or more sanitization devices like sanitizing device 100 and/or 101.

The sanitizing devices and systems disclosed herein may be used in a variety of situations to improve air quality and deactivate pathogens that may be present on surfaces and objects and/or in the air. For example, the sanitizing devices and systems disclosed herein may be used in common areas like transportation hubs, exercise facilities, schools, and medical facilities to sanitize surfaces and objects used by multiple individuals and the air in these common areas to reduce a likelihood that a person may come into contact with the pathogen and become ill. The sanitizing devices and systems disclosed herein may also be used to sanitize air flowing through heating and air conditioning systems in, for example, office buildings, restaurants, schools, museums, and/or sporting arenas.

In some embodiments the sanitizing devices and systems disclosed herein may be coupled with air filtration systems in order to, for example, work in conjunction with the air filtration systems to remove particles and/or deactivate pathogens in the air that may be present in the event of, for example, forest fires and/or a spring-time pollen bloom.

I claim:

1. A method for operating a sanitizing device, the method comprising:
initiating, by a processor resident within a control system for the sanitizing device, emission of sanitizing light into an environment, the control system being resident within the sanitizing device;
recording, by the processor, an operation of the sanitizing device while the sanitizing device is emitting sanitizing light into the environment; and
communicating, by the processor, the operation to an external computing device.

2. The method for claim 1, further comprising:
storing, by the processor, the operation in a memory resident within the sanitizing device.

3. The method for claim 1, wherein the sanitizing device is a first sanitizing device, the method further comprising:
establishing, by the processor, communication with a second sanitizing device.

4. The method for claim 3, wherein the communication between the first and the second sanitizing device is via a mesh network.

5. The method for claim 1, further comprising:
receiving, by the processor, a measurement of air quality from an air quality sensor.

6. The method for claim 1, wherein the operation is at least one of a duration of emission of the sanitizing light by the sanitizing device, when the sanitizing device is emitting sanitizing light into the environment, and how much electrical current is being used by the sanitizing light when emitting sanitizing light into an environment.
